(19) United States
(12) Patent Application Publication
(10) Pub. No.: US 2024/0050354 A1
(43) Pub. Date: Feb. 15, 2024

(54) POLYGLUTAMIC ACID COMPOSITIONS AND METHODS OF USING

(71) Applicant: Bonafide Health, LLC, Harrison, NY (US)

(72) Inventor: James R. Komorowski, Trumbull, CT (US)

(21) Appl. No.: 18/381,087

(22) Filed: Oct. 17, 2023

Related U.S. Application Data
(63) Continuation-in-part of application No. 18/177,720, filed on Mar. 2, 2023.
(60) Provisional application No. 63/315,964, filed on Mar. 2, 2022.

(51) Int. Cl. A61K 8/64; A61Q 19/00; A61K 8/04

(52) U.S. Cl. CPC A61K 8/64; A61Q 19/007; A61K 8/042

(57) ABSTRACT

Disclosed herein are compositions comprising one or more polyglutamic acid compounds. The compositions are for treating, ameliorating, preventing, or reducing the symptoms of vaginal dryness and the symptoms associated with menopause. Also described herein are compositions for treating, ameliorating, preventing, or reducing other symptoms of skin dryness of a subject's body, limbs, hands, feet, neck, or face, in people of all ages and genders. Also described herein are topical and suppository formulations of the compositions.

### CROSS-REFERENCE TO RELATED APPLICATIONS

This application is a continuation-in-part of U.S. patent application Ser. No. 18/177,720, filed Mar. 2, 2023, which claims priority to U.S. Provisional Application No. 63/315,964 filed Mar. 2, 2022, the disclosure of each of which is incorporated herein by reference in its entirety.

### BACKGROUND

Vaginal dryness is a common condition that can affect women of all ages. However, vaginal dryness is most common in women following menopause. In postmenopausal women, vaginal dryness may be associated with declining estrogen levels. Topical estrogen therapy has been the most common treatment for vaginal dryness, but this therapy carries risks such as vaginal bleeding and increased risk of uterine cancer. An alternative therapy for patients unwilling or unable to consider an estrogen therapy is hyaluronic acid. Yet, it also has adverse effect including vulvovaginal candidiasis and bacterial vaginosis. As such, the prior art methods and compositions for treatment of vaginal dryness are insufficient and solutions are provided herein to help alleviate vaginal dryness.

Skin dryness is another common condition that can affect women of all ages. Skin dryness also may occur or the frequency or severity of skin dryness may increase in women following menopause. In postmenopausal women, skin dryness may be associated with declining estrogen levels.

Other symptoms of skin dryness can affect people of all ages and genders.

### SUMMARY

Some embodiments of the present disclosure relate to novel compositions containing one or more polyglutamic acid compounds and their use in the treatment and/or amelioration of the symptoms of vaginal dryness and the symptoms associated with menopause.

Some embodiments of the present disclosure relate to novel compositions containing one or more polyglutamic acid compounds and their use in the treatment and/or amelioration of the symptoms of skin dryness, such as skin dryness associated with menopause. In some embodiments, compositions containing one or more polyglutamic acid compounds described herein are used to maintain healthy levels of skin dryness, including maintaining healthy levels of skin dryness compared to that which existed before menopause.

Some embodiments of the present disclosure relate to novel compositions containing one or more polyglutamic acid compounds and their use in the treatment and/or amelioration of the symptoms of skin dryness, such as skin dryness unrelated to menopause or declining estrogen levels.

Some embodiments of the present disclosure relate to novel compositions containing one or more polyglutamic acid compounds and their use in the treatment and/or amelioration of other symptoms of skin dryness in people of all ages and genders, such as other symptoms of skin dryness of a subject's body, limbs, hands, feet, neck, or face. In some embodiments, compositions containing one or more polyglutamic acid compounds described herein are used to maintain healthy levels of skin dryness of a subject's body, limbs, hands, feet, neck, or face.

These and other features, aspects, and advantages of the present embodiments will become understood with reference to the following description and appended claims.

### DETAILED DESCRIPTION

Described herein are compositions comprising one or more polyglutamic acid compounds and a pharmaceutically acceptable carrier. Each polyglutamic acid compound can be prepared by reacting a glutamic ester monomer with a suitable initiator. The molecular weight of each polyglutamic acid compound is determined by the number of the glutamic acid monomers that have been linked, corresponding to an n value set forth below.

The structure of a polyglutamic acid compound is depicted below:

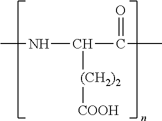

N refers to a chain length of a polyglutamic acid compound and can be between 300 and 30,000. The molecular weight of a polyglutamic acid compound of the disclosure can be between about 50 and 4,000 kDa. The composition described herein can contain one or more polyglutamic acid compounds that can have different polymer chain lengths (i.e., different n values).

For example, and without limitation, a composition can be comprised of a first polyglutamic acid compound, wherein the first polyglutamic acid compound has an n value of between about 300 and 500; a second polyglutamic acid compound, wherein the second polyglutamic acid compound has an n value between about 1,000 and 10,000; and a third polyglutamic acid compound, wherein the third polyglutamic acid compound has an n value between about 20,000 and 30,000.

The number of polyglutamic acid compounds having different n values in a composition is not particularly limited. In some embodiments, a composition can comprise one, two, three, four, five, six, or more different polyglutamic acid compounds that have different n values.

Without being bound to any particular theory, the compositions containing one or more polyglutamic acid compounds of the instant disclosure produce unexpectedly superior results due to the compounds' ability to penetrate varying layers of vaginal epithelial tissue depending upon the particular ratio of polyglutamic acid compounds having different n values. For example, a composition can comprise a first polyglutamic acid compound and a second polyglutamic acid compound, wherein the first polyglutamic acid compound has an n value that is less than the second polyglutamic acid compound. It is believed that a polyglutamic acid compound having a smaller n value is able to penetrate into deeper layers of the vaginal epithelium than a polyglutamic acid compound with a larger n value. And it is further believed that a second polyglutamic acid compound having an n value greater than a first polyglutamic acid compound will not penetrate into deeper epithelial layers, but rather, the second polyglutamic acid compound will stay closer to the surface than the first polyglutamic acid compound.

Certain embodiments comprise a first polyglutamic acid compound, a second polyglutamic acid compound, and a third polyglutamic acid compound. In certain embodiments, the first polyglutamic acid compound is a polyglutamic acid compound comprising a greater number of glutamic acid monomers than the second polyglutamic acid compound. The second polyglutamic acid compound comprises a greater number of glutamic acid monomers than the third polyglutamic acid compound. In some embodiments, the first polyglutamic acid compound is configured to penetrate into the stratus corneum layer of a subject's vaginal wall, the second polyglutamic acid compound is configured to penetrate into the stratum granulosum layer of a subject's vaginal wall, and the third polyglutamic acid compound is configured to penetrate into the stratum spinosum layer of a subject's vaginal wall. In some embodiments, one or more of the polyglutamic acid compounds can have an n value large enough such that it does not penetrate any skin layers, but stays on the skin surface, and can act as a lubricant.

Additionally or alternatively, and again without being bound to any particular theory, the present invention recognizes and has found that compositions containing one or more polyglutamic acid compounds of the instant disclosure produce unexpectedly superior results due to the compounds' ability to penetrate varying layers of skin tissue, such as layers of the epidermis and/or dermis of skin tissue of a subject's body, limbs, hands, feet, neck, or face, depending upon the particular ratio of polyglutamic acid compounds having different n values. For example, a composition can comprise a first polyglutamic acid compound and a second polyglutamic acid compound, wherein the first polyglutamic acid compound has an n value that is less than the second polyglutamic acid compound. It is believed that a polyglutamic acid compound having a smaller n value is able to penetrate into deeper epidermal and/or dermal layers of the skin than a polyglutamic acid compound with a larger n value. And it is further believed that a second polyglutamic acid compound having an n value greater than a first polyglutamic acid compound will not penetrate into deeper epidermal and/or dermal layers of the skin, but rather, the second polyglutamic acid compound will stay closer to the surface than the first polyglutamic acid compound.

Other exemplary embodiments comprise a first polyglutamic acid compound, a second polyglutamic acid compound, a third polyglutamic acid compound, a fourth polyglutamic acid compound, and/or a fifth polyglutamic acid compound. In certain embodiments, the first polyglutamic acid compound is a polyglutamic acid compound comprising a greater number of glutamic acid monomers than the second polyglutamic acid compound. The second polyglutamic acid compound comprises a greater number of glutamic acid monomers than the third polyglutamic acid compound. The third polyglutamic acid compound comprises a greater number of glutamic acid monomers than the fourth polyglutamic acid compound. The fourth polyglutamic acid compound comprises a greater number of glutamic acid monomers than the fifth polyglutamic acid compound.

In example embodiments, the first polyglutamic acid compound is configured to penetrate into layers of the epidermis including the stratum corneum, the second polyglutamic acid compound is configured to penetrate into the stratum lucidum, the third polyglutamic acid compound is configured to penetrate into the stratum granulosum, the fourth polyglutamic acid compound is configured to penetrate into the stratum spinosum, and/or the fifth polyglutamic acid compound is configured to penetrate into the stratum basale.

In example embodiments, the first polyglutamic acid compound is configured to penetrate into layers of the epidermis including one of the stratum corneum, the stratum lucidum, the stratum granulosum, the stratum spinosum, and the stratum basale, and the second polyglutamic acid compound is configured to penetrate into layers of the epidermis including another of the stratum corneum, the stratum lucidum, the stratum granulosum, the stratum spinosum, and the stratum basale.

In some embodiments, one or more of the polyglutamic acid compounds can have an n value large enough such that it does not penetrate any epidermal skin layers, but stays on the skin surface, such as on the surface of the stratum corneum, and can act as a lubricant.

In other example embodiments, the first polyglutamic acid compound is configured to penetrate into a layer of the epidermis and the second polyglutamic acid compound is configured to penetrate into a layer of the dermis, such as the papillary dermis and/or the reticular dermis.

In still other example embodiments, the first polyglutamic acid compound is configured to penetrate into a first layer of the dermis and the second polyglutamic acid compound is configured to penetrate into a second layer of the dermis.

The amount of each polyglutamic acid compound in any of the compositions described herein is not particularly limited, and may be about 10 μg to about 10 g. For example, the amount can be 10 μg, 15 μg, 20 μg, 25 μg, 30 μg, 35 μg, 40 μg, 45 μg, 50 μg, 55 μg, 60 μg, 65 μg, 70 μg, 75 μg, 80 μg, 85 μg, 90 μg, 95 μg, 100 μg, 125 μg, 150 μg, 175 μg, 200 μg, 225 μg, 250 μg, 275 ag, 300 μg, 325 μg, 350 μg, 375 μg, 400 μg, 425 μg, 450 μg, 475 μg, 500 μg, 525 μg, 575 μg, 600 ag, 625 μg, 650 μg, 675 μg, 700 μg, 725 μg, 750 μg, 775 μg, 800 μg, 825 μg, 850 μg, 875 μg, 900 ag, 925 μg, 950 μg, 975 μg, 1000 μg, 5 mg, 10 mg, 15 mg, 20 mg, 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, 100 mg, 125 mg, 150 mg, 175 mg, 200 mg, 225 mg, 250 mg, 275 mg, 300 mg, 325 mg, 350 mg, 375 mg, 400 mg, 425 mg, 450 mg, 475 mg, 500 mg, 525 mg, 575 mg, 600 mg, 625 mg, 650 mg, 675 mg, 700 mg, 725 mg, 750 mg, 775 mg, 800 mg, 825 mg, 850 mg, 875 mg, 900 mg, 925 mg, 950 mg, 975 mg, 1000 mg, 1.25 g, 1.5 g, 1.75 g, 2.0 g, 2.25 g, 2.5 g, 2.75 g, 3.0 g, 3.25 g, 3.5 g, 3.5 g, 3.75 g, 4.0 g, 4.25 g, 4.5 g, 4.75 g, 5.0 g, 5.25 g, 5.5 g, 5.75 g, 6.0 g, 6.25 g, 6.5 g, 6.75 g, 7.0 g, 7.25 g, 7.5 g, 7.75 g, 8.0 g, 8.25 g, 8.5 g, 8.75 g, 9.0 g, 8.25 g, 9.5 g, 9.75 g, 10 g, or any amount therebetween.

The compositions disclosed herein can be used to treat, prevent, or alleviate vaginal dryness, vaginal itching, and/or vaginal burning. In some embodiments, the compositions described herein can be used to maintain healthy levels of vaginal moisture. In some embodiments, the compositions described herein can be used to accelerate the rate of wound healing, prevent urinary tract infections, and/or maintain pH values in a vaginal tissue. In certain embodiments, a composition as described herein is administered to a subject to avoid painful intercourse. In certain embodiments, a composition as described herein is administered to a subject to rejuvenate a vaginal tissue.

In certain embodiments, a composition can be administered to a subject's vaginal tissue to treat, prevent, ameliorate, and/or reduce the symptoms of vaginal dryness. In certain embodiments, a composition can be administered to a subject's vaginal tissue to maintain a healthy level of vaginal moisture. In certain embodiments, a composition can be administered to a subject's vaginal tissue to treat, prevent, and/or ameliorate the symptoms of vaginal dryness due to menopause. In some embodiments, a composition can be administered to a subject's vaginal tissue to treat, prevent, and/or ameliorate one or more symptoms of menopause. In certain embodiments, a composition can be administered to a subject's vaginal tissue to reduce the pain associated with sexual activity, including sexual intercourse. In certain embodiments, a composition can be administered to a subject's vaginal tissue to treat, prevent, and/or ameliorate vaginal itching. In certain embodiments, a composition can be administered to a subject's vaginal tissue to treat, prevent, and/or ameliorate vaginal burning. In certain embodiments, a composition can be used for one or more of the foregoing.

Additionally, or alternatively, the compositions disclosed herein can be used to treat, prevent, or alleviate skin dryness, skin itching, and/or skin burning of a subject, such as a subject's body, limbs, hands, feet, neck, and/or face. In some embodiments, the compositions described herein can be used to maintain healthy levels of skin moisture. In some embodiments, the compositions described herein can be used to accelerate the rate of wound healing, prevent infections, and/or maintain pH values in a skin tissue. In certain embodiments, a composition as described herein is administered to a subject to rejuvenate skin tissue.

In certain embodiments, a composition can be administered to a subject's skin tissue, such as a subject's body, limbs, hands, feet, neck, and/or face, to treat, prevent, ameliorate, and/or reduce the symptoms of skin dryness. In certain embodiments, a composition can be administered to a subject's skin tissue, such as a subject's body, limbs, hands, feet, neck, and/or face, to maintain a healthy level of moisture. In certain embodiments, a composition can be administered to a subject's skin tissue, such as a subject's body, limbs, hands, feet, neck, and/or face, to treat, prevent, and/or ameliorate the symptoms of skin dryness due to menopause. In other embodiments, a composition can be administered to a subject's skin tissue, such as a subject's body, limbs, hands, feet, neck, and/or face, to treat, prevent, and/or ameliorate other symptoms of skin dryness in people of all ages and genders, such as other symptoms of skin dryness of a subject's body, limbs, hands, feet, neck, or face. In certain embodiments, a composition can be administered to a subject's skin tissue, such as a subject's body, limbs, hands, feet, neck, and/or face, to treat, prevent, and/or ameliorate skin itching. In certain embodiments, a composition can be administered to a subject's skin tissue, such as a subject's body, limbs, hands, feet, neck, and/or face, to treat, prevent, and/or ameliorate skin burning. In certain embodiments, a composition can be used for one or more of the foregoing.

In certain embodiments, a composition can be administered to a subject's skin tissue, such as a subject's body, limbs, hands, feet, neck, and/or face, to create a barrier to prevent moisture from leaving the subject's skin and/or to prevent dryness and irritation by locking moisture into the subject's skin. In some embodiments, a composition can be administered to a subject's skin tissue, such as a subject's body, limbs, hands, feet, neck, and/or face, to protect the subject's skin from environmental damage and/or to protect the subject's skin from allergy and infection-causing germs like bacteria and fungi. In still other embodiments, a composition can be administered to a subject's skin tissue, such as a subject's body, limbs, hands, feet, neck, and/or face, to promote anti-aging by keeping the subject's skin moist and supple.

In some embodiments, the compositions disclosed herein are in the form of pharmaceutically effective salts. The phrase “pharmaceutically acceptable salt(s),” is art recognized and, as used herein includes, but is not limited to, salts of acidic or basic groups that may be present in the compositions disclosed herein. Compounds that are basic in nature are capable of forming a wide variety of salts with various inorganic and organic acids. The acids that may be used to prepare pharmaceutically acceptable acid addition salts of such basic compounds are those that form non-toxic acid addition salts, i.e., salts containing pharmacologically acceptable anions, including but not limited to sulfuric, citric, maleic, acetic, oxalic, hydrochloride, hydrobromide, hydroiodide, nitrate, sulfate, bisulfate, phosphate, acid phosphate, isonicotinate, acetate, lactate, salicylate, citrate, acid citrate, tartrate, oleate, tannate, pantothenate, bitartrate, ascorbate, succinate, maleate, gentisinate, fumarate, gluconate, glycerate, glucaronate, saccharate, formate, benzoate, glutamate, methanesulfonate, ethanesulfonate, benzenesulfonate, p-toluenesulfonate and pamoate (i.e., 1,1′-methylene-bis-(2-hydroxy-3-naphthoate)) salts. Compounds present in the compositions disclosed herein that include an amino moiety also can form pharmaceutically acceptable salts with various amino acids, in addition to the acids mentioned above. Compounds present in the compositions disclosed herein that are acidic in nature are capable of forming base salts with various pharmacologically acceptable cations. Non limiting examples of such salts include alkali metal or alkaline earth metal salts and, particularly, calcium, magnesium, sodium lithium, zinc, potassium, silicon, phosphorus, and iron salts.

The phrase “pharmaceutically acceptable carrier” is art-recognized and includes a pharmaceutically acceptable material, composition or vehicle, suitable for administering compounds of the present invention to mammals. The carriers include liquid or solid filler, diluent, excipient, solvent or encapsulating material, involved in carrying or transporting the subject agent from one organ, or portion of the body, to another organ, or portion of the body.

Each carrier must be “acceptable” in the sense of being compatible with the other ingredients of the formulation and not injurious to the patient. Some examples of materials which can serve as pharmaceutically acceptable carriers include: sugars, such as lactose, glucose and sucrose; starches, such as corn starch and potato starch; cellulose and its derivatives, such as sodium carboxymethyl cellulose, ethyl cellulose, and cellulose acetate; powdered tragacanth; malt; gelatin; talc; excipients, such as cocoa butter and suppository waxes; oils, such as peanut oil, cottonseed oil, safflower oil, sesame oil, olive oil, corn oil, and soybean oil; glycols, such as propylene glycol; polyols, such as glycerol, glycerides, sorbitol, mannitol, and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; and other non-toxic compatible substances employed in pharmaceutical formulations. In one embodiment, the pharmaceutically acceptable carrier is suitable for intravenous administration. In another embodiment, the pharmaceutically acceptable carrier is suitable for locoregional injection.

The term “pharmaceutically acceptable esters” refers to the relatively non-toxic, esterified products of the compounds of the present invention. These esters can be prepared in situ during the final isolation and purification of the compounds, or by separately reacting the purified compound in its free acid form or hydroxyl with a suitable esterifying agent. Carboxylic acids can be converted into esters via treatment with an alcohol in the presence of a catalyst. Hydroxyls can be converted into esters via treatment with an esterifying agent such as alkanoyl halides. The term also includes lower hydrocarbon groups capable of being solvated under physiological conditions, e.g., alkyl esters, methyl, ethyl, and propyl esters.

The dosage form of compositions described herein is not particularly limited. In certain embodiments, the composition is formulated as a topical, such as a cream or an ointment. In certain embodiments, the composition is formulated as a gel. In certain embodiments the composition is formulated as a suppository.

In certain embodiments, a suppository formulation comprises one or two polyglutamic acid compounds, a solubilizer, a humectant, a surfactant, and/or one or more suppository bases. An emollient can also be included in a suppository formulation. An example of a solubilizer is water. An example of a humectant is glycerin. An example of a surfactant is polysorbate 80. Examples of suppository bases are PEG4000, Gelucire 43/01, PEG3350, witepsol W-35®, and saturated fatty acids glycerides C₁₂-C₁₈. An example of an emollient is softisan 378. The one or two polyglutamic acid compounds have molecular weights ranging from 50 kDa to 2000 kDa. For examples, the molecular weight of a polyglutamic acid compound of the compositions disclosed herein can be 50 kDa, 100 kDa, 150 kDa, 200 kDa, 250 kDa, 300 kDa, 350 kDa, 400 kDa, 450 kDa, 500 kDa, 550 kDa, 600 kDa, 650 kDa, 700 kDa, 750 kDa, 800 kDa, 850 kDa, 900 kDa, 950 kDa, 1000 kDa, 1050 kDa, 1100 kDa, 1150 kDa, 1200 kDa, 1250 kDa, 1300 kDa, 1350 kDa, 1400 kDa, 1450 kDa, 1500 kDa, 1550 kDa, 1600 kDa, 1650 kDa, 1700 kDa, 1750 kDa, 1800 kDa, 1850 kDa, 1900 kDa, 1950 kDa, or 2000 kDa. The weight percentage (% w/w) of each polyglutamic acid compound in any of the compositions described herein is not particularly limited and can range from about 0.01% to about 1%. For example, it can be 0.01%, 0.02%, 0.03%, 0.04%, 0.05%, 0.06%, 0.07%, 0.08%, 0.09%, 0.10%, 0.11%, 0.12%, 0.13%, 0.14%, 0.15%, 0.16%, 0.17%, 0.18%, 0.19%, 0.20%, 0.21%, 0.22%, 0.23%, 0.24%, 0.25%, 0.26%, 0.27%, 0.28%, 0.29%, 0.30%, 0.31%, 0.32%, 0.33%, 0.34%, 0.35%, 0.36%, 0.37%, 0.38%, 0.39%, 0.40%, 0.41%, 0.42%, 0.43%, 0.44%, 0.45%, 0.46%, 0.47%, 0.48%, 0.49%, 0.50%, 0.51%, 0.52%, 0.53%, 0.54%, 0.55%, 0.56%, 0.57%, 0.58%, 0.59%, 0.60%, 0.61%, 0.62%, 0.63%, 0.64%, 0.65%, 0.66%, 0.67%, 0.68%, 0.69%, 0.70%, 0.71%, 0.72%, 0.73%, 0.74%, 0.75%, 0.76%, 0.77%, 0.78%, 0.79%, 0.80%, 0.81%, 0.82%, 0.83%, 0.84%, 0.85%, 0.86%, 0.87%, 0.88%, 0.89%, 0.90%, 0.91%, 0.92%, 0.93%, 0.94%, 0.95%, 0.96%, 0.97%, 0.98%, 0.99%, 1%, or more, or any value therebetween. The weight percentages (% w/w) of the solubilizer, the humectant, the surfactant, the one or more suppository bases, and the emollient in the suppository formulation are also not particularly limited and can range, respectively, from 1% to 100%, from about 0.1% to 11.4%, from 0.1% to 1.4%, from 0% to 100%, and from 0% to 100%. The amount of the polyglutamic acid compounds, the solubilizer, the humectant, the surfactant, the suppository bases and/or the emollient are in a synergistic ratio. As used herein, a “synergistic ratio” refers to a ratio that elicits an unexpectedly superior pharmacological and physiological effect in a subject. A non-limiting example of the synergistic ratio of the amount of the polyglutamic acid compound to the amount of the solubilizer to the amount of the humectant to the amount of the surfactant, and to the amount of the suppository base is 5:396:50:28:1521. A non-limiting example of the synergistic ratio of the amount of the polyglutamic acid compound to the amount of the solubilizer to the amount of the humectant to the amount of the surfactant, and to the amount of the topical formulation is 5:396:50:28:1521.

In certain embodiments, a suppository formulation comprises only one polyglutamic acid compound. In certain embodiments, the compound has a molecular weight of 100 kDa, 700 kDa, or 2000 kDa. In certain embodiments, a suppository formulation contains a solubilizer, a humectant, a surfactant, and a suppository base. In certain embodiments, the solubilizer is water, the humectant is glycerin, the surfactant is polysorbate 80, and the suppository base is saturated fatty acids glycerides C₁₂-C₁₈.

In certain embodiments, a suppository formulation comprises two polyglutamic acid compounds. In certain embodiments, the compounds have molecular weights of 100 kDa and 700 kDa.

In certain embodiments a topical formulation comprises one or more polyglutamic acid compounds. Some embodiments of a topical formulation comprise a first polyglutamic acid compound and a second polyglutamic acid compound. In some embodiments the first polyglutamic acid compound has a molecular weight between about 50-200 kDa. In some embodiments, the first polyglutamic acid compound has a molecular weight between about 50-100 kDa, 100-200 kDa, 50-150 kDa, 75-125 kDa, 50-125 kDa, 150-200 kDa, 100-175 kDa, and ranges therebetween. In some embodiments, the second polyglutamic acid compound has a molecular weight between about 500-900 kDa. In some embodiments, the second polyglutamic acid compound has a molecular weight between about 500-1000 kDa, 600-900 kDa, 700-900 kDa, 800-900 kDa, 500-600 kDa, 500-700 kDa, 500-800 kDa, 500-900 kDa, 700-800 kDa, 550-750 kDa, 650-850 kDa and ranges therebetween. In some embodiments, a topical formulation can comprise a first polyglutamic acid compound and a second polyglutamic acid compound, wherein the first polyglutamic acid compound has a molecular weight of about 100 kDa, and the second polyglutamic acid compound has a molecular weight of about 700 kDa.

In certain embodiments, suppository formulations can be disintegrated between 5-10 minutes, between 10-20 minutes, or between 20-30 minutes. In certain embodiments, a suppository formulation can be administered every two days, every three days, every four days, every five days, every six days, every week, every eight days, every nine days, every ten days, every two weeks, every month, or more to provide fast and long-lasting relief of the symptoms of vaginal dryness and the symptoms associated with menopause.

In other embodiments, a topical formulation, such as a cream, ointment, or gel, can be administered multiple times per day, once per day, every two days, every three days, every four days, every five days, every six days, every week, every eight days, every nine days, every ten days, every two weeks, every month, or more to provide fast and long-lasting relief of the symptoms of skin dryness associated with menopause and/or other symptoms of skin dryness in people of all ages and genders, such as other symptoms of skin dryness of a subject's body, limbs, hands, feet, neck, or face. The frequency at which the topical formulation is to be applied is not particularly limited. In some embodiments, a topical formulation may be administered multiple times for day for several days in a row, without limitation. In some embodiments, a user may apply a topical formulation to skin to prophylactically prevent skin dryness. In certain embodiments, a user may apply a topical formulation to reduce the effects of skin dryness and moisturize skin. The topical formulation may be applied at a frequency that is effective to maintain healthy levels of skin moisture.

While the present invention has been described in some detail for purposes of clarity and understanding, one will appreciate that various changes in form and detail can be made without departing from the true scope of the invention.

### EXAMPLES

### Example 1

In one example, polyglutamic acid (mw: 100 kDa; 5 mg; 0.25% w/w) was mixed with water (396 mg; 19.8% w/w), glycerin (50 mg; 2.5% w/w), polysobrate 80 (28 mg; 1.4% w/w), and saturated fatty acids glycerides C₁₂-C₁₈ (1521 mg; 76.04% w/w) to yield a white suppository. A dissolution study of the suppository was conducted by using a phosphate buffer that has a pH range of about 4.2-4.5, which is within a range of normal vaginal pH (3.8-5.0). Percentages of polyglutamic acid in the buffer at hour 0.5, hour 0.75, hour 1, hour 2, hour 3, hour 6, hour 24, hour 30, and hour 48 were determined. The dissolution data are shown in Table 1 below.

TABLE 1
Dissolution Data

| Time | % Dissolved | | |
|---|---|---|---|
| (hr) | Average (n = 3) | Std Dev | % RSD |
| 0.5 | 67.16 | 13.24 | 19.7 |
| 0.75 | 68.27 | 19.91 | 29.2 |
| 1 | 72.97 | 19.12 | 26.2 |
| 2 | 88.21 | 9.90 | 11.2 |
| 3 | 98.09 | 1.78 | 1.8 |
| 4 | 100.01 | 4.19 | 4.2 |
| 24 | 101.83 | 3.25 | 3.2 |
| 30 | 100.64 | 3.09 | 3.1 |
| 48 | 102.85 | 3.13 | 3.0 |

It was observed that the suppository completely disintegrated within 30 minutes. The data shows that 67% of the polyglutamic acid compound was dissolved in 30 minutes and the polyglutamic acid compound was completely dissolved in the buffer at hour 3. Unexpectedly, a steady dissolution was observed from hour 0.5 to hour 3. Also unexpectedly, 100% of the polyglutamic acid compound remained in the buffer at hour 48, indicating that the compound is stable in a vaginal tissue and therefore, can moisturize the vaginal tissue for more than 2 days once administered. These unexpected results demonstrate that the composition can provide fast and long-lasting relief of the symptoms of vaginal dryness.

### Example 2

The vaginal suppository composition described in example 1 above will be studied to evaluate its short-term stability as compared to a placebo prototype composition. The placebo composition will be identical to the vaginal suppository composition except that the former does not contain any polyglutamic acid compound. Both compositions will be placed on ICH stability at 2-8° C. and 25° C./60% RH conditions for 3 months. At the end of month 1 and month 2, the stability of the vaginal suppository composition will be evaluated by visual appearance and the amount of the polyglutamic acid compound remained in the composition. At the end of month 3, the stability of the vaginal suppository composition will be evaluated by, in addition to visual appearance and the amount of polyglutamic acid remaining in the composition, the amount of the polyglutamic acid compound dissolved. As to the placebo composition, its stability will be evaluated by visual appearance and placebo interference at the end of each month.

### Example 3

An assay will be conducted where an in vitro vaginal tissue test apparatus will be used to test the activity and efficacy of varying polymer lengths of polyglutamic acid compounds. The varying polyglutamic acid compounds (i.e., with different n values) will be applied to the test apparatus, and their ability to penetrate into the various tissue layers of the vaginal wall will be correlated with their n value (i.e., the number of monomers in the polyglutamic acid compound), and the relative effect on vaginal moisture. In addition, compositions having varying concentrations of polyglutamic acid compounds with different polymer chain lengths (i.e., different n values) will be tested to ascertain the effect on vaginal moisture and penetration into vaginal tissue layers.

### Example 4

Certain compositions described herein will be tested for their effects on the vaginal epithelium in a postmenopausal rat model.

Thirty female Wistar/Sprague rats (eight-week-old) will be purchased from the Firat University Experimental Animal Center. All rats will be individually housed under clean conditions with controlled temperature, humidity, and light (12-h light-dark cycle) and provided a standard commercial diet and water ad libitum. All experimental procedures will follow the guidelines established by the Use of Laboratory Animals and will be approved by the Animal Care and Use Committee of Firat University.

Rats that exhibited regular 4-days oestrous cycle will be chosen for the experiment. The rats will be divided into seven groups as shown in Table 2. After one adaptation period, rats will be ovariectomized (OVX) or given a sham operation (control) under anesthesia. The same weight of adipose tissue will be removed from rats in the control group. After the surgery, the vaginal smears will be assessed daily. Only rats whose vaginal smears that have exhibited regular 4-days cycles (before and after sham/OVX) and rats that exhibited constant leukocytes (after OVX) will be used (10 rats for each group). The rest of rats will be dosed intra-vaginally or orally with the same volume of placebo (vehicle) gel as rats that received a composition. The body weights of rats will be measured weekly.

TABLE 2
Study groups

| | | | Animal dose (per rat/day) |
|---|---|---|---|
| Groups | Source | HED | (n = 10 per arm)* |
| 1 | Saline | — | — |
| 2 | OVX | — | |
| 3 | OVX+ | 5 mg | 0.51 mg/kg BW |
| 4 | OVX+ | 50 mg | 5.14 mg/kg BW |
| 5 | OVX+ | 500 mg | 51.42 mg/kg BW |
| 6 | OVX+ | 5000 mg | 514.16 mg/kg BW |
| 7 | OVX+ | 50 g | 0.52 g/kg BW |

*Doses of products were converted by allometric scaling, which takes into account differences in body surface area to calculate equivalent doses for rat by the following calculation, assuming an 60 kg human: RD = HED(g)/60kg*6.17 (For example: (50 mg /60)*6.17=5.14 mg/kg BW); where RD is the rat dosage of products, HED is the human equivalent dosage of products, and 6.17 is the conversion factor to convert a human dosage to a rat dosage

On day 15, the treatment effect of each composition for vaginal atrophy will be determined by a vaginal smear obtained from each rat, with a histological examination of a vaginal tissue. Vaginal smears will be fixed and detected, the expression of leukocyte esterase, β-glucuronidase, and coagulase will be used to determine the effect of each composition on vaginal microecosystem. Then all rats will be sacrificed by cervical dislocation and their vagina and uterus will be removed. The wet weight of each uterus will be determined once they will be carefully cleaned of any adherent tissue. To minimize variability due to dissection, all uteri will be dissected above the cervix at the same distance. The vaginal tissue will be then removed and will be cut into 1 mm×1 mm×2 mm sections, stained with 2.5% glutaraldehyde. Some vaginal samples will be taken and processed for histological examination by standard procedures (4-5 μm sections stained with haematoxylin plus eosin).

The repair capacity of each composition will be detected by Western blot. Vaginal VEGF, p-AKT, COX-2, ERα, and ERβ will be detected by Western blot method. For Western blot, proteins will be separated in 10% polyacrylamide gel, electrotransferred topolyvinylidene fluoride membranes, and the bands will be blocked in 5% dried milk for 1 hour at room temperature and incubated with different primary antibodies (1:1000). The incubation will be carried out in PBS supplemented with 5% skim milk powder. After washing with Tris-Buffered Saline (TBS) containing 0.1% Tween-20 (TBS-T), the membranes will be further incubated with an anti-rabbit IgG antibody conjugated with horseradish peroxidase at room temperature for 1 hour. After washing with TBS-T, antigen-antibody complex will be visualized by using an enhanced-chemiluminescence-detection kit.

### Example 5

An assay will be conducted where a skin tissue test apparatus will be used to test the activity and efficacy of varying polymer lengths of polyglutamic acid compounds. The varying polyglutamic acid compounds (i.e., with different n values) will be applied to the test apparatus, and their ability to penetrate into the various tissue layers of the skin will be correlated with their n value (i.e., the number of monomers in the polyglutamic acid compound), and the relative effect on skin moisture. In addition, compositions having varying concentrations of polyglutamic acid compounds with different polymer chain lengths (i.e., different n values) will be tested to ascertain the effect on skin moisture and penetration into skin tissue layers.

1. A composition for treating skin dryness comprising one or more polyglutamic acid compounds and a pharmaceutically acceptable carrier.

2. The composition of claim 1, wherein the one or more polyglutamic acid compounds comprises a first polyglutamic acid compound and a second polyglutamic acid compound.

3. The composition of claim 2, further comprising a third polyglutamic acid compound.

4. The composition of claim 3, further comprising a fourth polyglutamic acid compound.

5. The composition of claim 4, further comprising a fifth polyglutamic acid compound.

6. The composition of claim 2, wherein the second polyglutamic acid compound has an n value that is greater than an n value of the first polyglutamic acid compound, wherein the n value is the number of the glutamic acid monomers linked together to form each polyglutamic acid compound.

7. The composition of claim 3, wherein the second polyglutamic acid compound has an n value that is greater than an n value of the first polyglutamic acid compound, and the third polyglutamic acid compound has an n value that is greater than the n value of the second polyglutamic acid compound, wherein the n value is the number of the glutamic acid monomers linked together to form each polyglutamic acid compound.

8. The composition of claim 4, wherein the second polyglutamic acid compound has an n value that is greater than an n value of the first polyglutamic acid compound, the third polyglutamic acid compound has an n value that is greater than the n value of the second polyglutamic acid compound, and the fourth polyglutamic acid compound has an n value that is greater than the n value of the third polyglutamic acid compound, wherein the n value is the number of the glutamic acid monomers linked together to form each polyglutamic acid compound.

9. The composition of claim 5, wherein the second polyglutamic acid compound has an n value that is greater than an n value of the first polyglutamic acid compound, the third polyglutamic acid compound has an n value that is greater than the n value of the second polyglutamic acid compound, the fourth polyglutamic acid compound has an n value that is greater than the n value of the third polyglutamic acid compound, and the fifth polyglutamic acid compound has an n value that is greater than the n value of the fourth polyglutamic acid compound, wherein the n value is the number of the glutamic acid monomers linked together to form each polyglutamic acid compound.

10. The composition of claim 1, wherein the composition is a topical formulation.

11. The composition of claim 10, wherein the topical formulation is a cream.

12. The composition of claim 10, wherein the topical formulation is an ointment.

13. The composition of claim 10, wherein the topical formulation is a gel.

14. The composition of claim 6, wherein the first polyglutamic acid compound has a molecular weight of 100 kDa and the second polyglutamic acid compound has a molecular weight of 700 kDa.

15. A method of treating skin dryness, comprising administering a composition of claim 1 to a subject's skin.

16. The method of claim 15, wherein the composition is administered to the subject's body, limbs, hands, feet, neck, and/or face.

17. The method of claim 16, wherein the composition is a topical formulation.

18. The method of claim 17, wherein the topical formulation is one of a cream, an ointment, and a gel.

19. A method for maintaining a healthy level of skin moisture, comprising administering a composition of claim 1 to a subject's skin tissue.

20. The method of claim 19, wherein the composition is administered to the subject's body, limbs, hands, feet, neck, and/or face.

21. The method of claim 20, wherein the composition is a topical formulation.

22. The method of claim 21, wherein the topical formulation is one of a cream, an ointment, and a gel.

23. A method for treating, preventing, and/or ameliorating one or more symptoms of menopause, comprising administering a composition of claim 1 to a subject's skin tissue.

24. The method of claim 23, wherein the symptoms are skin dryness, skin itching, and/or skin burning.

25. The method of claim 24, wherein the composition is administered to the subject's body, limbs, hands, feet, neck, and/or face.

26. The method of claim 25, wherein the composition is a topical formulation.

27. The method of claim 25, wherein the topical formulation is one of a cream, an ointment, and a gel.
